(19) United States
(12) Patent Application Publication
(10) Pub. No.: US 2005/0267426 A1
(43) Pub. Date: Dec. 1, 2005

(54) Male incontinence device

(76) Inventor: Jethro Shedrick, Washington, DC (US)

Correspondence Address: BARNES & THORNBURG, SUITE 900, 750 17TH STREET, N.W., WASHINGTON, DC, 20006-4607, US

(21) Appl. No.: 10/854,178

(22) Filed: May 27, 2004

(51) Int. Cl. A61F 13/15

(52) U.S. Cl. 604/347; 604/385.09

(57) ABSTRACT

The present disclosure relates to male incontinence devices attachable to an undergarment and including a moisture absorbent pad.

### BACKGROUND

The present disclosure relates to devices dealing with male incontinence. More particularly, it relates to an absorbent pad attachable to an undergarment.

Incontinence affects a great number of males of all ages. Some cancers or surgeries are responsible for this. Many males simply cannot restrain or control their need to urinate. A number of approaches have been tried to provide males with a means to contain or absorb urinary discharges without leakages and an embarrassing staining of their undergarments. Some of those devices have been either too bulky and discomforting or they inhibit a user's ability to go through the process of normal urination without revealing the existence of the incontinence device to others. Therefore, for a male to lead a relatively normal life, it is desirable that an incontinence device enable him to have the protection of a moisture absorbent pad while still permitting a normal urination process to take place.

### SUMMARY

The present disclosure, therefore, relates to a male incontinence device for use with a user's undergarment. The device includes a moisture absorbent pad having at least one moisture permeable surface and a holder attachable to the user's undergarment. The holder has an opening to releasably receive at least a portion of the moisture absorbent pad. After the moisture absorbent pad is received in the holder, an end portion of the pad is located immediately adjacent a penile opening in the undergarment and/or immediately adjacent an edge of the undergarment configured to be used as a penile opening.

The moisture absorbent pad may have at least one moisture impermeable surface. The holder may have at least one moisture impermeable surface.

Other aspects of the present disclosure will become apparent from the following descriptions when considered in conjunction with the accompanying drawings.

### BRIEF DESCRIPTION OF THE DRAWINGS

FIG. 1 is a perspective view of an embodiment of a male incontinence device including a holder mounted on an inner surface of a portion of a user's undergarment, and the device having a moisture absorbent pad in the holder with one outer essentially planar surface being moisture permeable and the other opposite essentially planar surface being moisture impermeable.

FIG. 2 is a perspective view of another embodiment of a male incontinence device including a holder mounted on an inner surface of a portion of a user's undergarment, and the device having a moisture absorbent pad with one outer, essentially planer surface being completely moisture permeable and the other opposite essentially planar surface being partially moisture impermeable, and an outer surface of the holder that is facing the user's undergarment being moisture impermeable.

FIG. 3 is a perspective view of another embodiment of a male incontinence device mounted on an inner surface of a portion of a user's undergarment, and the device having a moisture absorbent pad with both essentially planar surfaces being moisture permeable and an outer, essentially planar surface of the holder that is facing the user's undergarment being moisture impermeable.

FIG. 4 is a perspective view of an embodiment of a male incontinence device including a holder mounted on an inner surface of a portion of a user's undergarment, the holder including vertically-mounted strap-like elements, and the device having a moisture absorbent pad in the holder with one outer essentially planar surface being moisture permeable and the other opposite planar surface being moisture impermeable.

FIG. 5 is a perspective view of an embodiment of a male incontinence device similar to FIG. 4, but with the strap-like elements of the holder being mounted horizontally.

### DETAILED DESCRIPTION

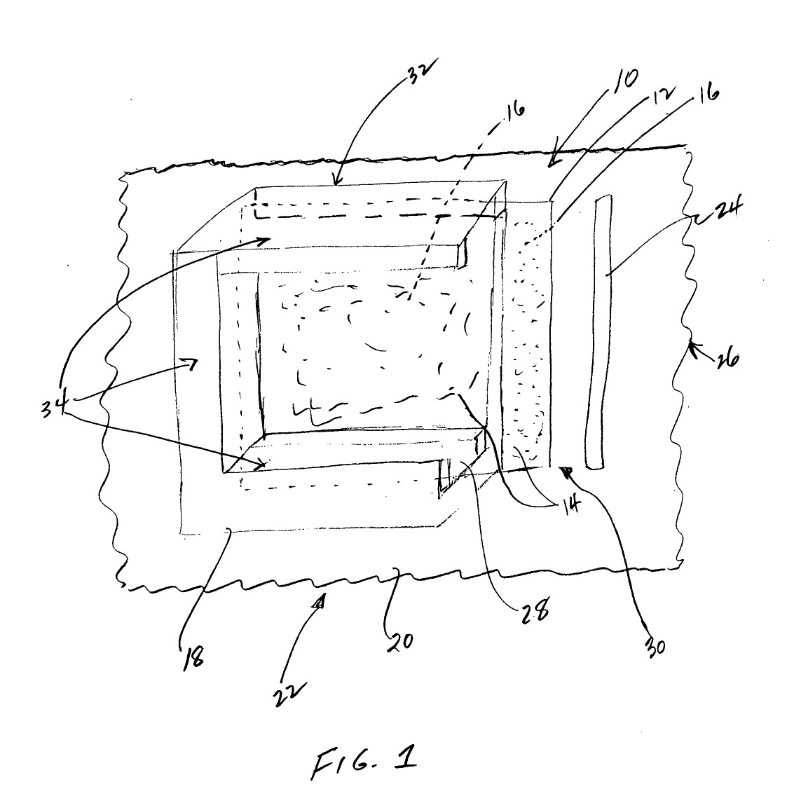

An embodiment of a male incontinence device 10 is shown in FIG. 1. The device 10 includes a moisture absorbent pad 12 having at least one moisture permeable surface 14. The pad 12 may also have a moisture impermeable surface 16 located opposite from the moisture permeable surface 14, as shown in FIG. 1. The device 10 also includes a holder 18 which is attachable to a front inner surface 20 of a user's undergarment 22, as shown in FIG. 1. The holder 18 is attached offset from one or both of a penile opening 24 in the undergarment 22 or an edge 26 of the undergarment 22 that is configured to be used as a penile opening. The holder 18 has an opening 28 to receive at least a portion of the pad 12. Pad 12 extends exterior to the holder 18 and an end 30 of the pad 12 is located immediately adjacent the penile opening 24 or the edge 26 of the undergarment 22 configured to be used as a penile opening. In FIG. 1, moisture permeable surface 14 faces toward the front of the user's body (the front of the user's body, for reference, is facing the Figures, just as it is for the reader) and moisture impermeable surface 16 faces away from the user's body and toward the front inner surface 20 of the user's undergarment 22.

The holder 18 has an outer surface 32 that may be moisture permeable or moisture impermeable. The holder 18 may be of various shapes and sizes, and is shown in FIG. 1 having a picture frame-type configuration with slots 34 on three sides of the holder 18. The outer surface or surfaces 32 of the holder 18, as shown in FIG. 1, may be either moisture permeable or moisture impermeable since the side of the pad 12 facing the undergarment 22 has a moisture impermeable surface 16. Other holder 18 configurations are conceivable, such as a pocket having one opening, not shown, or other equivalent structures configured to receive complementary-structured pads 12.

The holder 18 may be releasably attached to the undergarment 22 using, for example, buttons and button holes (not shown), or adhesion means, such as a hook and loop arrangement (not shown). The holder may be permanently attached to the undergarment 22 using, for example, adhesives, sewing or ironing. Attachment by ironing is shown in FIGS. 1-3.

The pads 12 generally have two, essentially planar or possibly somewhat rounded surfaces sealed together at their ends. The pads 12 may have one moisture permeable side or surface 12 and one moisture impermeable side or surface 12. The pads 12 may have two moisture permeable surfaces 14 or two moisture impermeable surfaces 16. Or, the pads 12 may have one or both surfaces being partially permeable and partially impermeable.

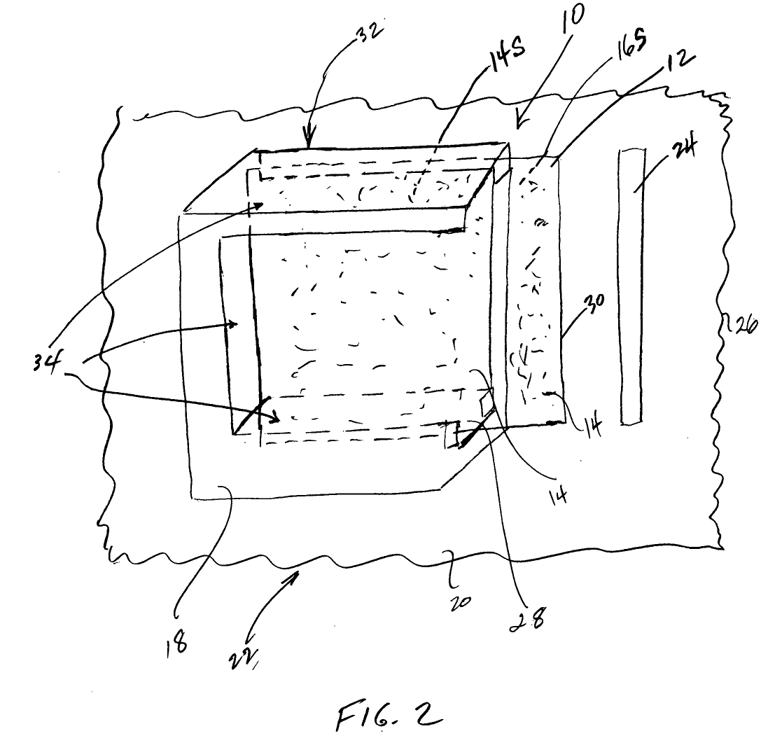

As shown in the embodiment of FIG. 2, the moisture absorbent pad 12 of device 10 is configured such that it includes a surface having moisture impermeable section 16S and a moisture permeable section 14S facing toward the inner surface 20 of undergarment 22. The pad 12 further includes a moisture permeable surface 14 facing toward the front of the user's body. The holder 18 in this embodiment is configured such that outer surface 32 covers the complete side of the holder 18 that is attached to the front inner surface 20 of the undergarment 22. The outer surface 32 in this embodiment is moisture impermeable. As in the embodiment of FIG. 1, the pad 12 extends exterior to the holder 12 and the end 30 of the pad 12 is located immediately adjacent the penile opening 24 or the edge 26 of the undergarment 22 configured to be used as a penile opening.

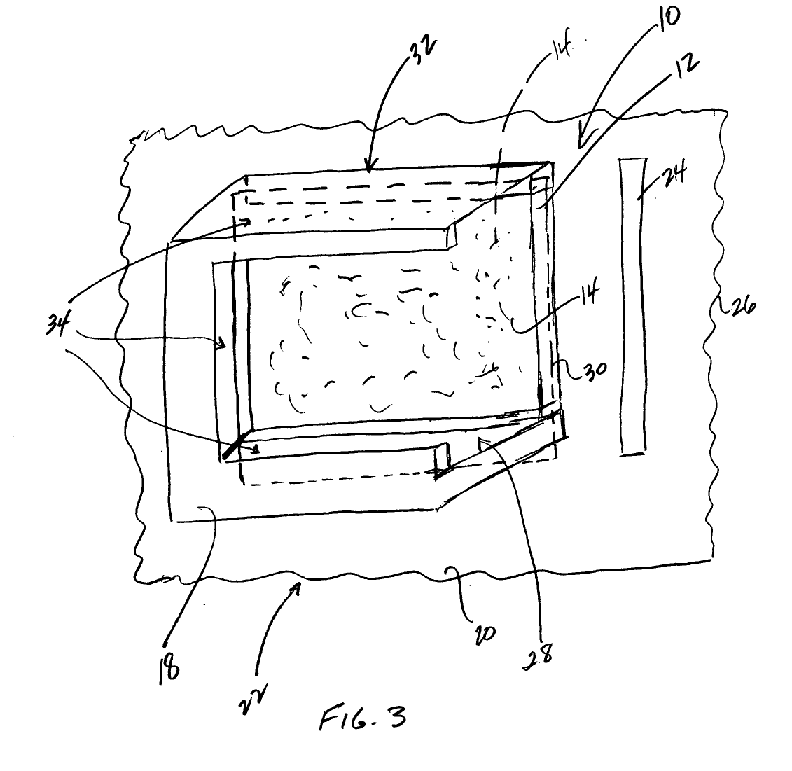

In another embodiment of the device 10, shown in FIG. 3, the pad 12 has two moisture permeable surfaces 14. In this embodiment, the holder 18 has an outer surface 32 attached to the front inner surface 20 of the undergarment 22 that is completely moisture impermeable and the pad 12 is essentially completely contained within the holder 18. The pad 12 is positioned such that the end 30 is located immediately adjacent the penile opening 24 or the edge 26 of the undergarment 22 configured to be used as a penile opening.

The holder 18 may be attached such that he holder opening 28 faces toward the left or right side of the user's body. The opening 28 may be oriented essentially horizontal and transverse to a user's height, as shown on FIGS. 1-3. However, the opening 28 may be oriented vertically (not shown).

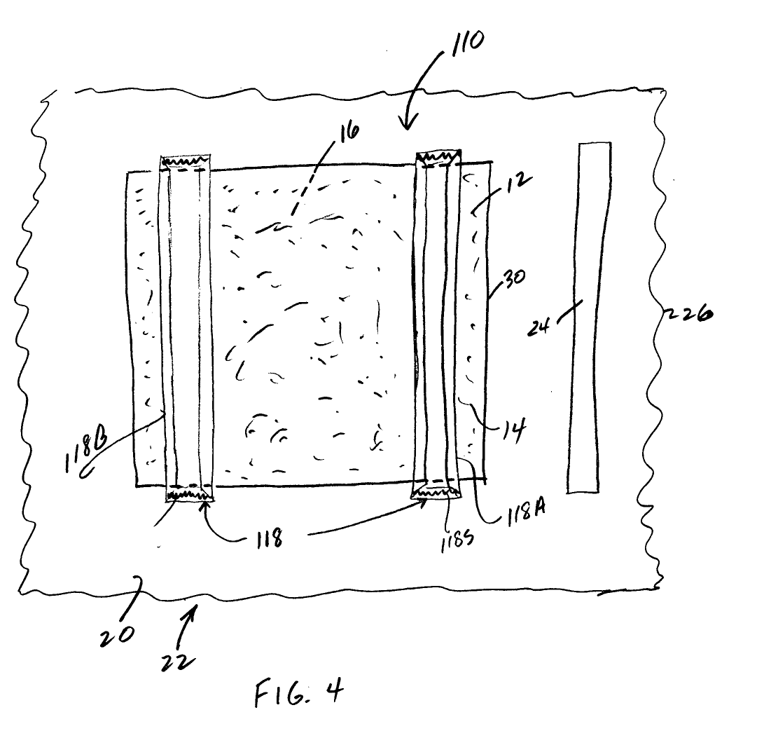

Another embodiment of a male incontinence device 110 is shown in FIG. 4. The device 110 includes a moisture absorbent pad 12 having at least one moisture permeable surface 14 and at least one moisture impermeable surface 16 located opposite from the moisture permeable surface 16. The device 110 also includes a holder 118 which is attachable to a front inner surface 20 of a user's undergarment 22. The holder 118 includes at least two strap-like elements 118A and 118B which may, for example, be sewn to the undergarment 22, as shown at 118S in FIG. 4. Other means of attachment of the strap-like elements are conceivable, such as a hook and loop arrangement, glue or other suitable means.

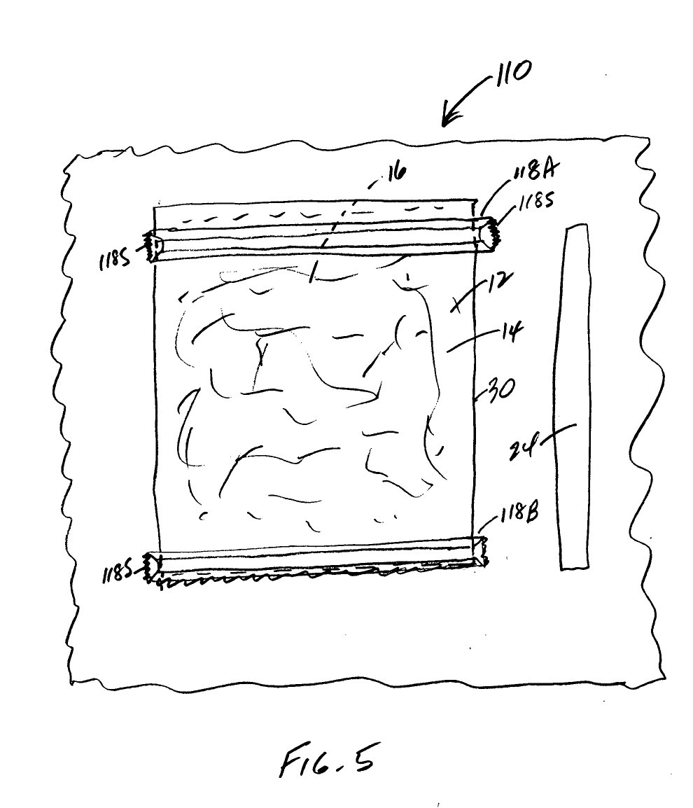

The strap-like elements 118A, B may be attached in a vertical manner, as shown in FIG. 4 or may be attached in a horizontal manner with the lower strap 118B being attached or sewn 118S such that the inserted pad 12 will not fall out, as shown in FIG. 5.

The strap-like elements 18A, B are constructed such that their surfaces may be at least partially moisture impermeable and they may be partially or totally made of elastic or elastic-type material.

Although the present disclosure has been described and illustrated in detail, it is to be clearly understood that this is done by way of illustration and example only and is not to be taken by way of limitation. The spirit and scope of the present disclosure are to be limited only by the terms of the appended claims.

1. A male incontinence device for use with a user's undergarment, comprising:
a moisture absorbent pad including at least one moisture permeable surface;
a holder attachable to an inner surface of the user's undergarment and offset from at least one of a penile opening of the undergarment and an edge of the undergarment configured to be used as a penile opening, the holder having an opening to receive at least a portion of the moisture absorbent pad; and
wherein after the moisture absorbent pad is received in the holder, an end of the pad is located immediately adjacent at least one of the penile opening in the undergarment and the edge of the undergarment configured to be used as a penile opening.

2. The device of claim 1, wherein the moisture absorbent pad further includes a moisture impermeable surface located opposite from the at least one moisture permeable surface.

3. The device of claim 1, wherein the moisture absorbent pad further includes another moisture permeable surface located opposite from the at least one moisture permeable surface.

4. The device of claim 1, wherein the moisture absorbent pad further includes a moisture impermeable section located on at least a portion of a surface located opposite from the at least one moisture permeable surface.

5. The device of claim 4, wherein the moisture absorbent pad is inserted in the opening of the holder such that the at least one moisture permeable surface faces toward the front of the user's body and the moisture impermeable surface faces away from the front of the user's body and toward a front inner surface of the user's undergarment.

6. The device of claim 4, wherein the holder includes a moisture impermeable surface.

7. The device of claim 6, wherein the moisture impermeable section covers only an end section of the surface located opposite from the at least one moisture permeable surface, and the pad is inserted in the opening of the holder such that the moisture permeable surface faces toward the front of the user's body and the surface having the moisture impermeable section faces toward the moisture impermeable surface of the holder.

8. The device of claim 3, wherein the holder includes a moisture impermeable surface and the pad is inserted in the opening of the holder such that one of the pad's moisture permeable surfaces faces toward the front of the user's body and the other faces toward the moisture impermeable surface of the holder.

9. The device of claim 1, wherein the holder is releasably attached to the user's undergarment.

10. The device of claim 1, wherein the holder is attached to the user's undergarment such that the holder opening is oriented toward the left side of the user's body.

11. The device of claim 1, wherein the holder is attached to the user's undergarment such that the holder opening is oriented toward the right side of the user's body.

12. The device of claim 1, wherein the holder is attached to the user's undergarment such that the opening is oriented essentially horizontal and transverse to the user's height.

13. The device of claim 1, wherein the holder is attached by ironing, such that the ironing adheres at least a portion of an outer surface of the holder having heat sensitive adhesive to a portion of a front inner surface of the user's undergarment.

14. The device of claim 1, wherein the holder is attached by sewing at least a portion of an outer surface of the holder to at least a portion of a front inner surface of the user's garment.

15. The device of claim 1, wherein the holder includes adhesion means couplable to at least a portion of a front inner surface of the user's undergarment, such front inner surface having complementary adhesion means.

16. The device of claim 15, wherein the adhesion means is one of hook and loop construction and the complementary adhesion means is one of hook and loop construction.

17. The device of claim 1, wherein the holder is formed as a picture-frame type holder having slots configured to receive at least a portion of the moisture absorbent pad.

18. The device of claim 1, wherein the holder includes at least two strap-like elements.

19. The device of claim 18 wherein the strap-like elements are mounted vertically.

20. A male incontinence device for use with a user's undergarment, the device comprising:
a moisture absorbent means;
a receiving means for receiving at least a portion of the moisture absorbent means, the receiving means being attachable to at least a portion of a front inner surface of a user's undergarment and offset from at least one of a penile opening of the undergarment and an edge of the undergarment configured to be used as a penile opening; and
wherein the moisture absorbent means is inserted in an opening of the receiving means such that an end of the moisture absorbent means is located immediately adjacent at least one of the penile opening in the undergarment and the edge of the undergarment configured to be used as a penile opening.

21. The device of claim 18, wherein the receiving means is a holder having a slot on three sides to releasably retain the moisture absorbent means.

22. The device of claim 18, wherein the holder is attached to the user's undergarment such that the opening is oriented essentially horizontal and the opening faces toward one of the user's right and left.
